(19) United States
(12) Patent Application Publication
(10) Pub. No.: US 2020/0086147 A1
(43) Pub. Date: Mar. 19, 2020

(54) COMPOSITION OF DIGESTIVE ENZYMES AND APPETITE SUPPRESSANTS

(71) Applicant: Ernest Joon Kim, Huntington Beach, CA (US)

(72) Inventor: Ernest Joon Kim, Huntington Beach, CA (US)

(21) Appl. No.: 16/130,394

(22) Filed: Sep. 13, 2018

(51) Int. Cl. A61P 1/14; A61P 3/04

(52) U.S. Cl. CPC A61P 1/14; C12Y 302/01176; C12Y 302/01001; C12Y 204/01004; C12Y 304/11001; C12Y 302/01048; C12Y 302/01015; C12Y 302/01003; C12Y 302/01004; C12Y 302/01032; C12Y 302/01022; C12Y 304/21; C12Y 301/01003; C12Y 302/01026; C12Y 302/01108; A61P 3/04

(57) ABSTRACT

The present invention relates to a composition, particularly a composition comprising digestive enzymes and appetite suppressant. The composition can be used for reducing appetite, inducing weight loss and/or promote food digestion in a subject in need thereof.

### FIELD OF THE INVENTION

The present invention relates to a composition, particularly a composition comprising a digestive enzyme and an appetite suppressant.

### BACKGROUND OF THE INVENTION

Many people have difficulty in digesting food. With the advancement of age, the human body produces fewer enzymes to break down food and thus has more difficulty with digestion. Compounding the problem, our modern diet of processed and refined foods can add stress to our digestive system and prematurely weaken it.

To aid in the digestion of food, some people take digestive enzyme supplements. Digestive enzymes help break down food and improve digestion, but may tend to make people feel hungry again after the food was digested. By speeding up digestion, people may overeat, digest their food, and then tend to overeat again because they feel hungry sooner. This may cause the person to eat more than they otherwise would eat, which may cause weight gain and contribute to obesity.

Obesity is among the top health concerns in the United States. It is not healthy when the body accumulates excess body fat, which in turn causes health risks such as high blood pressure, diabetes, heart disease, stroke, and so forth. Persons become obese when they consume too many calories and are not active enough to burn those calories. Persons who eat too much will gain weight, and if appetite is suppressed, then persons will lose weight because they eat less.

Natural appetite suppressants are substances, foods, or herbs that help keep persons from overeating. Natural appetite suppressants may help reduce overeating, in part, by balancing levels of hunger hormones, normalizing blood sugar levels, or even improving mood so there is less overeating from stress or depression.

There is a need to develop a composition that can speed up the digestion of food through enzymes while suppressing appetite.

### SUMMARY OF THE INVENTION

One aspect of the invention is to provide a composition comprising a digestive enzyme and an appetite suppressant.

In an embodiment of the invention, the digestive enzyme is selected from the group consisting of protease, lipase, amylase, invertase, lactase, cellulase, xylanase, aspergillopepsin, α-galactosidase, β-Glucanase, glucoamylase, pectinase, peptidase, aminopeptidase, cholecystokinin, secretin, sucrase, maltase, isomaltase, elastase, trypsin, chymotrypsin, nuclease, phospholipase, pepsin, and lysozyme.

In another embodiment of the invention, the digestive enzyme is coated with a polymer selected from the group consisting of cellulose acetate phthalate, hydroxypropylmethylcellulose phthalate, hydroxypropylmethylcellulose acetate succinate, shellac, acrylic acid polymer, methacrylic acid polymer, and methacrylic acid/methylmethacrylate copolymer.

In another embodiment of the invention, the appetite suppressant is selected from the group consisting of naringin, saffron extract, green tea extract, garcinia cambogia, 5-Hydroxy-L-tryptophan (5-HTP), cap saicin, black pepper, turmeric, ginger, and cinnamon.

In another embodiment of the invention, the composition further comprises a stabilizer selected from the group consisting of proline, trehalose, dextran, maltose, sucrose, mannitol, polyols, silica gel, aminoguanidine, pyridoxamine, anhydrous metal salts, magnesium oxide, calcium oxide, aluminum oxide, and mixtures thereof.

In another embodiment of the invention, the composition further comprising a binder selected from the group consisting of starches, sugars, lactose, sugar alcohols, xylitol, sorbitol, maltitol, cellulose, microcrystalline cellulose, modified celluloses, hydroxypropylcellulose, carboxymethylcellulose sodium, alginic acid, polyvinyl pyrrolidone, and mixtures thereof.

In another embodiment of the invention, the composition is in the form of liquid, capsules, microcapsules, tablets, granules, powder, troches, pills, suppositories, injections, suspensions, syrups or effervescent.

In another embodiment of the invention, the composition further comprises a pharmaceutically acceptable carrier.

Another aspect of the invention is to provide a method for reducing appetite in a subject in need thereof.

Another aspect of the invention is to provide a method for reducing appetite in a subject in need thereof, comprising administering to the subject an amount of the composition of the invention.

Another aspect of the invention is to provide a method for inducing weight loss in a subject in need thereof, comprising administering to the subject an amount of the composition of the invention.

Another aspect of the invention is to provide a method for reducing appetite and promoting food digestion in a subject in need thereof, comprising administering to the subject an amount of the composition of the invention.

### DETAILED DESCRIPTION OF THE INVENTION

In the following sections, various exemplary compositions and methods are described in order to detail various embodiments of the invention. It will be obvious to one skilled in the art that combining and synthesizing the various embodiments does not require the employment of all or even some of the details outlined herein, but rather that concentrations, times and other details may be modified through routine experimentation. In some cases, well known methods, or components have not been included in the description.

### Definitions

Unless otherwise defined herein, scientific and technical terms used in connection with the present invention shall have the meaning commonly understood by those of ordinary skill in the art. The meaning and scope of the terms should be clear; however, in the event of any latent ambiguity, definitions provided herein take precedence over any dictionary or extrinsic definition.

Unless otherwise required by context, singular terms shall include the plural and plural terms shall include the singular. For example, the term “a” or “an,” as used herein, is defined as one or more than one.

As used herein, the term “or” in the claims is used to mean “and/or” unless explicitly indicated to refer to alternatives only or the alternatives are mutually exclusive.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein the term “about” refers to ±30%, preferably±20%, more preferably ±10%, and even more preferable ±5%.

As used herein, the term “composition” refers to liquid, capsules, microcapsules, tablets, granules, powder, troches, pills, suppositories, injections, suspensions, syrups, effervescent. The composition can be administered to a subject orally or parenterally.

As used herein, the term “digestive enzyme” refers to an enzyme in the alimentary tract which breaks down the components of food so that they can be taken or absorbed by the organism. Digestive enzyme includes but is not limited to pancrelipase, pancreatin, lipase, trypsin, chymotrypsin, chymotrypsin B, pancreatopeptidase, carboxypeptidase A, carboxypeptidase B, glycerol ester hydrolase, phospholipase, sterol ester hydrolase, elastase, kininogenase, ribonuclease, deoxyribonuclease, a-amylase, papain, chymopapain, glutenase, bromelain, ficin, β-amylase, cellulase, β-galactosidase, lactase, sucrose and isomaltase.

As used herein, the term “appetite suppressant” refers to a substance or an agent that induces feelings of satiety and/or tricks the body into believing it is not hungry. Appetite suppressant includes but is not limited to african mango (Irvingia gabonensis), beta-glucans, bitter orange (Citrus aurantium L.), caffeine, calcium, capsaicin, capsaicinoids, carnitine, chitosan, chromium, coleus forskohlii, conjugated linoleic acid, fucoxanthin, garcinia cambogia (hydroxycitric acid), glucomannan, green coffee bean extract, green tea (Camellia sinensis) extract, saffron extract, guar gum, hoodia (hoodia gordonii), pyruvate, raspberry ketone, vitamin

D, white kidney bean (Phaseolus vulgaris), Griffonia Simplicifolia and yohimbe (Pausinystalia yohimbe).

As used herein, the term “polymer” refers to a large molecule, or macromolecule, composed of many repeated subunits. The polymer includes but is not limited to cellulose acetate phthalate, hydroxypropylmethylcellulose phthalate, hydroxypropylmethylcellulose acetate succinate, shellac, acrylic acid polymers, methacrylic acid polymers, and methacrylic acid/methylmethacrylate copolymers.

As used herein, the term “stabilizer” refers to a substance that enhances or improves the stability of the compositions of the invention. The polymer includes but is not limited to proline, trehalose, dextran, maltose, sucrose, mannitol, polyols, silica gel, aminoguanidine, pyridoxamine, anhydrous metal salts, magnesium oxide, calcium oxide, aluminum oxide, and mixtures thereof.

As used herein, the term “binder” refers to a substance that ensures the tablets and granules being formed with required mechanical strength, and give volume to low active dose tablets. The polymer includes but is not limited to starches, sugars, lactose, sugar alcohols, xylitol, sorbitol, maltitol, cellulose, microcrystalline cellulose, modified celluloses, hydroxypropylcellulose, carboxymethylcellulose sodium, alginic acid, polyvinyl pyrrolidone, and mixtures thereof.

As used herein, the term “pharmaceutically acceptable carrier” refers to solvents, diluents, binders, adhesives, adjuvants, excipients, acceptors, stabilizer, analogues, flavoring agents, sweetening agents, emulsifying agents or preservative agents, which are well known to persons of ordinary skill in the art, for manufacturing pharmaceutical or dietary compositions. Examples of pharmaceutically acceptable carriers include, but are not limited to, water, saline, buffers, and inert, nontoxic solids.

As used herein, the term “administering” or “administration” refers to the methods that may be used to enable delivery of the composition or medicament of the present invention to the desired site of biological action. These methods include, but are not limited to, oral, intraduodenal, nasal, parenteral injection (including intravenous, subcutaneous, intramuscular, intravascular or intradermal), topical and rectal administration.

As used herein, the term “therapeutically effective amount” refers to the amount of composition which, when administered to a human or animal, is sufficient to result in a therapeutic effect in said human or animal.

As used herein, the term “treatment,” “treat” or “treating” refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, decreasing the rate of disease progression, amelioration and palliation of the disease state.

As used herein, the term “delaying development of a disease” means to defer, hinder, slow, retard, stabilize, suppress and/or postpone development of the disease. This delay can be of varying lengths of time, depending on the history of the disease and/or individual being treated.

As used herein, the term “subject” is a mammal, such as a human or other animal, and typically is human. In some embodiments, the subject has been treated with a therapeutic agent targeting the disease or condition prior to administration.

### Composition Comprising Digestive Enzymes and Appetite Suppressants

The invention provides a composition comprising a digestive enzyme and an appetite suppressant.

Examples of the digestive enzyme include, but are not limited to, protease, lipase, amylase, invertase, lactase, cellulase, xylanase, aspergillopepsin, α-galactosidase, β-Glucanase, glucoamylase, pectinase, peptidase, aminopeptidase, cholecystokinin, secretin, sucrase, maltase, isomaltase, elastase, trypsin, chymotrypsin, nuclease, phospholipase, pepsin, and lysozyme.

Examples of the appetite suppressant include, but are not limited to, naringin, saffron extract, green tea extract, garcinia cambogia, 5-Hydroxy-L-tryptophan (5-HTP), capsaicin, black pepper, turmeric, ginger, and cinnamon.

An illustrative example of a composition comprises 10% to 75% by weight composition (w/w) of digestive enzyme and 10% to 78% by weight composition (w/w) of appetite suppressant, respectively.

In one embodiment, a digestive enzyme is present in an amount ranging from about 0.1 mg to about 1000 mg, about 0.5 mg to about 800 mg, about 1 mg to about 500 mg, about 5 mg to about 450 mg, about 20 mg to about 300 mg, or about 50 mg to about 250 mg by weight of the composition.

In a further embodiment, a digestive enzyme is present in an amount of about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, or about 1000 mg by weight of the composition.

In one embodiment, an appetite suppressant is present in an amount ranging from about 0.1 mg to about 10,000 mg, about 0.5 mg to about 9,000 mg, about 1 mg to about 5,000 mg, about 5 mg to about 4,000 mg, about 20 mg to about 3,000 mg, about 50 mg to about 2,500 mg about 80 mg to about 2,500 mg, or about 100 mg to about 2,000 mg by weight of the composition.

In a further embodiment, an appetite suppressant is present in an amount of about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 2,000 mg, about 3,000 mg, about 4,000 mg, about 5,000 mg, about 6,000 mg, about 7,000 mg, about 8,000 mg, about 9,000 mg or about 10,000 mg by weight of the composition.

Digestive enzymes and appetite suppressants are for different types of consumers. The consumers who take digestive enzymes are people who “eat so much” they are too full and may even feel sick, and the consumers who take appetite suppressants are trying to lose weight and are trying to “eat less”.

The invention combines the benefits of digestive enzymes which help in the digestion of food with the addition of an appetite suppressant, thereby helping a person control the amount of food that is consumed.

The composition of the invention optionally comprises a polymer that protects the digestive enzymes from gastric contents. The polymer used can be stable at acidic pH, but can break down rapidly at higher pH. For example, the polymer is insoluble in the pH range of from about 1.5 to about 5 (normally present in gastric fluids), and soluble at a pH of from about 6 to about 9, the normal pH range for mammalian intestinal fluids. The polymer includes but is not limited to cellulose acetate phthalate, hydroxypropylmethylcellulose phthalate, hydroxypropylmethylcellulose acetate succinate, shellac, acrylic acid polymers, methacrylic acid polymers, and methacrylic acid/methylmethacrylate copolymers.

The composition of the invention optionally comprises a stabilizer that enhances or improves the stability of the compositions of the invention. The polymer includes but is not limited to proline, trehalose, dextran, maltose, sucrose, mannitol, polyols, silica gel, aminoguanidine, pyridoxamine, anhydrous metal salts, magnesium oxide, calcium oxide, aluminum oxide, and mixtures thereof.

The composition of the invention optionally comprises a binder that ensures the tablets and granules being formed with required mechanical strength, and give volume to low active dose tablets. The polymer includes but is not limited to starches, sugars, lactose, sugar alcohols, xylitol, sorbitol, maltitol, cellulose, microcrystalline cellulose, modified celluloses, hydroxypropylcellulose, carboxymethylcellulose sodium, alginic acid, polyvinyl pyrrolidone, and mixtures thereof.

Examples of the compositions of the invention are food products including food supplement, functional food, and a dietary dupplement. A “food supplement” designates a product made from compounds usually used in foodstuffs, but which is in the form of tablets, powder, capsules, potion or any other form usually not associated with aliments, and which has beneficial effects for one's health. A “functional food” is an aliment which also has beneficial effects for one's health. In particular, food supplements and functional food can have a physiological effect—protective or curative—against a disease. If the composition according to the invention is a food product, it can be administered as such, can be mixed with a suitable drinkable liquid, such as water, yoghurt, milk or fruit juice, or can be mixed with solid or liquid food. In this context the dietary supplement or functional food can be in the form of tablets, pills, capsules, lozenges, granules, powders, suspensions, sachets, pastilles, effervescent mixtures, sweets, bars, syrups and corresponding administration forms, usually in the form of a unit dose. Preferably, the dietary supplement or functional food comprising the composition of the invention is administered in the form of tablets, lozenges, capsules or powders, manufactured in conventional processes of preparing dietary supplements.

The compositions described herein can be pharmaceutically acceptable compositions, which may include one or more pharmaceutically acceptable carriers, excipients, binders, diluents or the like. The instant compositions can be formulated for various routes of administration, for example, by oral administration. They also may be provided in combination with delivery vehicles such as in some encapsulating technology.

The compositions may be formulated for administration, in any convenient way for use in human intake. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The compositions of the invention can be prepared according to any method known to the art for the manufacture of pharmaceuticals, food or food supplements. Such compositions can contain sweetening agents, flavoring agents, coloring agents and preserving agents. A formulation can be admixtured with nontoxic acceptable excipients which are suitable for manufacture. Formulations may comprise one or more diluents, emulsifiers, preservatives, buffers, excipients, etc., and may be provided in such forms as liquids, powders, emulsions, lyophilized powders, tablets, pills or gels, etc. The composition for oral administration can be formulated using acceptable carriers well known in the art in appropriate and suitable dosages. Such carriers enable the compositions to be formulated in unit dosage forms as tablets, pills, powder, dragees, capsules, liquids, lozenges, gels, syrups, slurries, suspensions, etc., suitable for ingestion by the subject.

### Applications

The invention provides a method for reducing appetite in a subject in need thereof.

The invention also provides a method for reducing appetite in a subject in need thereof, comprising administering to the subject an amount of the composition of the invention.

The invention also provides a method for inducing weight loss in a subject in need thereof, comprising administering to the subject an amount of the composition of the invention.

The invention further provides a method for reducing appetite and promoting food digestion in a subject in need thereof, comprising administering to the subject an amount of the composition of the invention.

In one embodiment, the composition is in an amount ranging from about 350 mg to about 3,900 mg/kg, about 500 mg to about 3,000 mg/kg, about 800 mg to about 2,500 mg/kg, about 1,000 mg to about 2,000 mg/kg, or about 1,200 mg to about 1,500 mg/kg.

When digestive enzymes and appetite suppressants are combined, the result is a comprehensive and powerful supplement which aids in the digestion of food and helps avoid additional overeating. The advantages of the invention include, but are not limited to, the following benefits:
Speeding up the digestion of food through enzymes;
Promoting the absorption of nutrients;
Increasing thermogenesis and burning more calories; and
Suppressing appetite to prevent overeating.

The following examples are provided to aid those skilled in the art in practicing the present invention. Even so, the examples should not be construed to unduly limit the present invention as modifications to and variations on the embodiments discussed herein may be made by those having ordinary skill in the art without departing from the spirit or scope of the present inventive discovery.

### EXAMPLE 1

### Preparation of Digestive Enzymes

Digestive enzymes of the invention were commercially available and were prepared in the amount ranges as shown in Table 1 below.

TABLE 1

| Digestive Enzyme | Activity Units | Amount Range |
|---|---|---|
| Protease | 5,000 to 100,000 HUT (Hemoglobin Units, | about 20 mg to about 500 mg |
| | Tyrosine basis) | |
| Lipase | 20 to 4000 FTP (Fédération Internationale | about 5 mg to about 100 mg |
| | Pharmaceutique) | |
| Amylase | 80 to 50,000 DU (Dextrinizing Unit) | about 10 mg to about 450 mg |
| Invertase | 100 to 1000 SU (Summer Units) | about 2 mg to about 150 mg |
| Lactase | 400 to 2500 LU (Lactase Units) | about 10 mg to about 350 mg |
| Cellulase | 10 to 4000 CU (Cellulase Units) | about 2 mg to about 250 mg |
| Xylanase | 100 to 800 XU (Xylanase Units) | about 2 mg to about 150 mg |
| Aspergillopepsin | 50 to 500 SAPU (Spectrophotometric Acid | about 20 mg to about 350 mg |
| | Protease Unit) | |
| α-galactosidase | 50 to 750 GalU (Galactosidase Unit) | about 5 mg to about 150 mg |
| β-Glucanase | 20 to 150 BGU (Beta Glucanase Unit) | about 5 mg to about 150 mg |
| Glucoamylase | 10 to 200 AGU (Amyloglucosidase Units) | about 20 mg to about 300 mg |
| Pectinase | 10 to 150 edo-PGU (Endo Polygalacturonidase | about 5 mg to about 200 mg |
| | Unit) | |
| Peptidase | 3,000 to 40,000 HUT | about 20 mg to about 350 mg |
| Bromelain | 300,000 to 600,000 PU (Papain units) | about 100 mg to about |
| | 20 to 300 GDU (Gelatin Digesting) | 1200 mg |
| Papain | 5,000 to 1,000,000 PU | about 20 mg to about 100 mg |

### EXAMPLE 2

### Preparation of Digestive Enzyme Complex

Digestive enzyme complex was prepared in the amount ranges as shown in Tables 2 and 3 below.

TABLE 2

| Digestive Enzyme Complex | Amount per Serving |
|---|---|
| Bromelain | about 500,000 PU |
| Peptidase | about 8,000 HUT |
| Protease 3.0 | about 25 SAPU |
| Protease 4.5 | about 25,000 HUT |
| Invertase | about 200 SU |
| Amylase | about 10,000 DU |
| Cellulase | about 500 CU |
| Glucoamylase | about 20 AGU |
| Lactase | about 2500 LU |
| Lipase | about 40 FIP |

TABLE 3

| Digestive Enzyme Complex | Amount per Serving |
|---|---|
| Bromelain | about 120 GDU |
| Papain | about 600,000 PU |
| Protease 3.0 | about 10 SAPU |
| Protease 4.5 | about 400 HUT |
| Amylase | about 400 DU |
| Lipase | about 60 LU |
| Lactase | about 20 ALU |
| Cellulase | about 10 CU |
| Hemicellulase | about 10 HCU |

### EXAMPLE 3

### Preparation of Appetite Suppressants

Appetite suppressants of the invention were prepared in Table 3 below.

TABLE 3

| Appetite Suppressant | Amount Range |
|---|---|
| Naringin (from grapefruit extract) | about 20 mg to about 300 mg |
| Saffron extract | about 40 mg to about 350 mg |
| Green tea extract (standardized | about 100 mg to about 800 mg |
| epigallocatechin gallate (EGCG) | |
| extract) | |
| Garcinia Cambogia (with at least 10% | about 100 mg to about 2000 mg |
| HCA) | |
| 5-HTP | about 20 to about 600 mg |
| Capsaicin (in the range of 100-250,000 | about 20 mg to about 1500 mg |
| SHU (Scoville Heat Units)) | |
| Black pepper or black pepper extract | about 2 mg to about 500 mg |
| (with at least 10% piperine content) | |
| Turmeric (with at least 10% curcumin | about 20 mg to about 2500 mg |
| content) | |
| Ginger or Ginger extract | about 10 mg to about 9000 mg |
| Cinnamon | about 10 mg to about 3000 mg |

Efficacy of Green Tea Extract

Human trials have examined the effects of green tea extract on weight loss and weight maintenance. Human trials analyzed the results from 14 randomized controlled trials of green tea preparations in a total of 1,562 overweight or obese participants. The trials lasted from 12 to 13 weeks, and doses of green tea extract ranged from about 141 to about 1,207 mg. Green tea supplementation reduced body weight by a mean of 0.95 kg more than placebo.

Efficacy of Saffron Extract

In human trails, twice-daily, women subjects (n=60, overweight) were given 1 capsule of Saffron extract (about 176.5 mg/day) or an inactive placebo with no limitation in dietary intake. After 2 months, the subjects using the saffron extract had a decrease in snacking and lost more weight than the control group.

### EXAMPLE 3

### Preparation of a Composition Comprising Digestive Enzyme Complex and an Appetite Suppressant

A composition comprising a digestive enzyme and an appetite suppressant was prepared in Table 3 below.

TABLE 3

| Composition No. | Digestive Enzyme | Appetite Suppressant |
|---|---|---|
| 1 | Digestive Enzyme | Green Tea Extract |
| | Complex according | |
| | to Example 2 | |
| 2 | Digestive Enzyme | Saffron Extract |
| | Complex according | |
| | to Example 2 | |

Efficacy of the Composition Comprising Digestive Enzyme Complex and an Appetite Suppressant

There was a double-blind, placebo-controlled study using a randomized within-subject design to evaluate the effects of Composition 1 or Composition 2 of the invention in terms of food intake in grams and kilocalories and subsequent food ratings of hunger, fullness, prospective consumption, desire to eat and satisfaction pre dosing, pre and post meals and at hourly intervals across the day. Total mean number of grams (g) and kilocalories (kcal) consumed was measured.

The results of human trials showed that the subjects taking Composition 1 or Composition 2 had lost more weight than the subjects taking green tea extract only or saffron extract only. The results suggest that digestive enzyme complex may enhance the efficacy of the appetite suppressant.

Numerous modifications and variations of the invention as set forth in the above illustrative examples are expected to occur to those skilled in the art. Consequently, only such limitations as appear in the appended claims should be placed on the invention.

What is claimed is:

1. A composition comprising a digestive enzyme and an appetite suppressant.

2. The composition of claim 1, wherein the digestive enzyme is selected from the group consisting of protease, lipase, amylase, invertase, lactase, cellulase, xylanase, aspergillopepsin, α-galactosidase, β-Glucanase, glucoamylase, pectinase, peptidase, aminopeptidase, cholecystokinin, secretin, sucrase, maltase, isomaltase, elastase, trypsin, chymotrypsin, nuclease, phospholipase, pepsin, and lysozyme.

3. The composition of claim 1, wherein the digestive enzyme is further coated with a polymer selected from the group consisting of cellulose acetate phthalate, hydroxypropylmethylcellulose phthalate, hydroxypropylmethylcellulose acetate succinate, shellac, acrylic acid polymer, methacrylic acid polymer, and methacrylic acid/methylmethacrylate copolymer.

4. The composition of claim 1, wherein the appetite suppressant is selected from the group consisting of naringin, saffron extract, green tea extract, garcinia cambogia, 5-HTP, capsaicin, black pepper or black pepper extract, turmeric, ginger or ginger extract and cinnamon.

5. The composition of claim 1, further comprising a stabilizer selected from the group consisting of proline, trehalose, dextran, maltose, sucrose, mannitol, polyols, silica gel, aminoguanidine, pyridoxamine, anhydrous metal salts, magnesium oxide, calcium oxide, aluminum oxide, and mixtures thereof.

6. The composition of claim 1, further comprising a binder selected from the group consisting of starches, sugars, lactose, sugar alcohols, xylitol, sorbitol, maltitol, cellulose, microcrystalline cellulose, modified celluloses, hydroxypropylcellulose, carboxymethylcellulose sodium, alginic acid, polyvinyl pyrrolidone, and mixtures thereof.

7. The composition of claim 1, which is in the form of liquid, capsules, microcapsules, tablets, granules, powder, troches, pills, suppositories, injections, suspensions, syrups or effervescent.

8. The composition of claim 1, further comprising a pharmaceutically acceptable carrier.

9. A method for reducing appetite in a subject in need thereof, comprising administering to the subject an amount of the composition of claim 1.

10. A method for inducing weight loss in a subject in need thereof, comprising administering to the subject an amount of the composition of claim 1.

11. A method for reducing appetite and promoting food digestion in a subject in need thereof, comprising administering to the subject an amount of the composition of claim 1.
